(19) United States
(12) Patent Application Publication
(10) Pub. No.: US 2010/0222787 A1
(43) Pub. Date: Sep. 2, 2010

(54) TENSION CONTROL DEVICE

(75) Inventors: Louis B. Goode, Cranberry Township, PA (US); Robert Booker, Vandergrift, PA (US)

Correspondence Address: BRINKS HOFER GILSON & LIONE/INDY/COOK;BRINKS HOFER GILSON & LIONE, CAPITAL CENTER, SUITE 1100, 201 NORTH ILLINOIS STREET, INDIANAPOLIS, IN, 46204-4220, US

(73) Assignee: Cook Vascular Incorporated, Vandergrift, PA (US)

(21) Appl. No.: 12/712,360

(22) Filed: Feb. 25, 2010

Related U.S. Application Data
(60) Provisional application No. 61/156,611, filed on Mar. 2, 2009.

(51) Int. Cl. A61B 17/00

(52) U.S. Cl. 606/129

(57) ABSTRACT

A device for controlling a tensional force on an implanted elongated structure during removal of the implanted elongated structure from a body vessel of a patient via a removal tool. The removal tool is of a type that includes a sheath member having a passageway extending therethrough for receiving the elongated structure. A shaft member structured for engagement with the removal tool has a locking member disposed thereon. The locking member is constructed and arranged for selectively locking a position of the elongated structure in a manner to maintain a tension on the elongated structure, and unlocking the elongated structure to permit an adjustment of the tension.

### RELATED APPLICATION

The present patent document claims the benefit of the filing date under 35 U.S.C. §119(e) of Provisional U.S. Patent Application Ser. No. 61/156,611, filed Mar. 2, 2009, which is hereby incorporated by reference.

### BACKGROUND

1. Technical Field

This invention relates generally to devices for use in the medical arts. More particularly, the invention relates to a device for controlling the amount of tension exerted on a targeted elongated implanted structure, such as an electrical pacemaker or defibrillator lead, during removal of the elongated structure from a body vessel.

2. Background Information

A variety of medical treatments and surgical methods entail implanting an elongated structure in the body of a human or veterinary patient. Examples of such elongated structures include catheters, sheaths and cardiac electrical leads (such as pacemaker leads and defibrillator leads), as well as a variety of other devices. Over time, it can become necessary or desirable to remove the implanted elongated structure from the body of the patient. However, if the elongated structure has been implanted for an extended period of time, encapsulating biological tissue can grow around the elongated structure, making it difficult to remove the structure from the encapsulating tissue.

A heart pacemaker is typically implanted in a subcutaneous tissue pocket in the chest wall of a patient. A pacemaker lead extends from the pacemaker through a vein into a chamber of the patient's heart. The pacemaker lead commonly includes a conductor, such as an electrical wire coil, for conducting electrical signals (such as stimulating and/or sensing signals) between the pacemaker and the heart. Leads for defibrillators are generally similar to pacemaker leads, and are positioned about the heart. Defibrillator leads may be affixed either internally or externally of the heart.

While cardiac electrical leads typically have a useful life of many years, over time such leads may become encapsulated by fibrotic tissue against the heart itself or the wall of the vein, or against other surrounding tissue. Encapsulation is especially encountered in areas where the velocity of the flow of blood is low. The fibrotic tissue can be very tough, which makes it difficult to remove the lead from the area of the heart without causing trauma to the area. When small diameter veins through which a pacemaker lead passes become occluded with fibrotic tissue, separation of the lead from the vein can cause severe damage to the vein, including the possible dissection or perforation of the vein. In such cases, separation of the lead from the vein is usually not possible without restricting or containing movement of the lead, i.e., fixing the lead in position with respect to the patient, in particular, with respect to the patient's vein.

To avoid this and other possible complications, some inoperable cardiac leads are simply left in the patient when the pacemaker or defibrillator is removed or replaced. However, such a practice can incur the risk of an undetected lead thrombosis, which can result in stroke, heart attack, or pulmonary embolism. Such a practice can also impair heart function, as plural leads can restrict the heart valves through which they pass.

There are many other reasons why removal of an inoperable lead may be desirable. For example, if there are too many leads positioned in a vein, the vein can be obstructed to the extent that fluid flow through the vein is severely compromised. In addition, multiple leads can be incompatible with one another, thereby interfering with the pacing or defibrillating function. An inoperative lead can migrate during introduction of an adjacent second lead, and mechanically induce ventricular arrhythmia. Other potentially life-threatening complications can require the removal of the lead as well. For example, removal of an infected pacemaker lead may be desirable so as to avoid conditions such as septicemia or endocarditis.

Surgical removal of a heart lead in such circumstances may require open heart surgery. However, open heart surgery is accompanied by significant risk and cost to the patient, as well as a potential for unintended complications. A variety of methods and apparatuses have been devised as alternatives to open heart surgery for heart lead removal. Several of these methods and apparatuses are described in related patent documents, such as U.S. Pat. Nos. 5,697,936, 5,507,751, 5,632,749, 5,207,683, 4,943,289, 5,011,482, 5,013,310, 4,988,347, 5,423,806, 6,419,674, 6,687,548, 6,712,826, 7,359,756, and U.S. Pat. Publ. Nos. 2006/0235431, 2006/0253179, 2007/0191919, 2008/0071341, 2008/0071342, 2008/0147061, among others. Each of the aforementioned documents is incorporated by reference as if fully set forth herein.

Many of the aforementioned patent documents describe manual, or mechanical, devices that are used for removing an implanted structure, such as a pacemaker lead. Others describe non-mechanical techniques, such as laser extraction and radio frequency extraction. Non-mechanical techniques have been found effective in many cases when the amount and/or placement of fibrous growth that surrounds the implanted lead renders mechanical extraction difficult or impossible.

Although the devices described hereinabove have been found to be effective in removing many implanted leads and other indwelling structures from a vessel, difficulties may be encountered during certain stages of the removal process. One particularly problematic aspect involves the ability to maintain a sufficient tension on a lead, in order to facilitate grasping and removal of the lead. Once an exposed end of a lead has been severed from the cardiac device (e.g., pacemaker or defibrillator), a lead extender or locking stylet is often used to “grasp” the severed end of the implanted lead. A removal sheath as described in the referenced prior art documents is initially placed over the proximal end of the lead extender or locking stylet, and advanced in a forward (i.e., distal) direction. As the sheath is advanced into the vessel, a tensional force is preferably maintained on the lead or stylet to allow for safe advancement of the sheath. Maintaining this tensional force compels the sheath to follow and track-over the lead within the vessel along a path that limits undesired progression of the sheath toward, into, and potentially through the patient's vascular or cardiac wall.

However, applying this tensional force to the lead may be problematic for the physician. Typically, the physician's hands are occupied by manipulating the removal sheath(s) and/or operating the removal apparatus (e.g., laser, RF, or mechanical removal devices). In order to position the tip of these removal devices over the desired entry point into the vessel, the physician often needs to grasp the device close to the distal tip with one hand. Manipulation of the removal device, such as by pushing, turning, or activation of a power mechanism, may require the physician to grasp the proximal end of the device with the other hand. Therefore, use of both hands is typically required during operation of the lead removal device. As a result, in order to apply and maintain a tension to the lead extender or stylet during sheath advancement, the physician must rely on a scrub nurse or other trained medical professional to either apply this force, or to otherwise assist in the operation of the removal device to free up a hand of the physician in order to enable him/her to apply the force. In either event, the necessity to utilize the services of a second person in this manner removes a certain element of control from the physician. It also increases the cost and complexity of the lead removal procedure.

In addition to the foregoing, when the tensional force is applied, it is important that the proper amount of force be applied. Application of too little force may not provide sufficient tension to the lead to enable track-over of the sheath. Application of too much force may cause disengagement or splitting of a segment of the lead from the remainder of the implanted lead. This results in the removal of only the disengaged segment of the lead, thereby necessitating further action to remove the remainder of the lead from the vessel. In addition, the nurse or other assistant may not be in the best position to determine the proper amount of tension to be applied to the lead. While the physician may be in a better position to make this determination, the physician's hands are generally otherwise occupied on other aspects of the lead removal procedure, and are not necessarily available to assist in this task.

It is desired to provide a device for use in controlling tension on an implanted elongated structure targeted for removal. It is further desired that such device is capable of being positioned in a manner such that it is operable by the physician during the removal process, and does not require the assistance of a second person.

### SUMMARY

In one form thereof, the present invention comprises a device for controlling a tensional force on an implanted elongated structure during removal of the implanted elongated structure from a body vessel of a patient via a removal tool. The removal tool comprises a sheath member having a passageway extending therethrough. The sheath member is sized such that the elongated structure is receivable in the passageway and a proximal end of the elongated structure is extendable therethrough. The device comprises a shaft member structured for engagement with the removal tool. The shaft member has a locking member disposed thereon. The locking member is constructed and arranged for selectively locking a position of the elongated structure in a manner to maintain a tension on the elongated structure, and unlocking the elongated structure to permit an adjustment of the tension.

In another form thereof, the invention comprises a system for removing an elongated cardiac lead from a body vessel. The system includes a lead removal tool comprising an elongated sheath having a proximal end, a distal end, and a passageway extending therethrough. The sheath has a length such that at least a distal portion of the sheath is receivable in the body vessel. The passageway is sized such that the cardiac lead is receivable therein, and a proximal end of the cardiac lead is extendable therethrough. A tension control device is engaged with the lead removal tool. The tension control device comprises a shaft member having a proximal end and a distal end. An engagement member at the distal end is constructed and arranged for engagement with the lead removal tool. A locking member at the proximal end is constructed and arranged for receiving the extended cardiac lead proximal end and selectively locking a position of the cardiac lead in a manner to maintain a tension on the lead, and unlocking the position of the cardiac lead to permit an adjustment of the tension.

In still another form thereof, the invention comprises a method for controlling tension on an implanted elongated structure during removal of the implanted elongated structure from a body vessel of a patient. A tool is provided for removing the implanted elongated structure. The tool comprises a sheath having a proximal end, a distal end, and a passageway extending therethrough, wherein the sheath is sized such that the elongated structure is receivable in the passageway. A device is provided for engagement with the tool. The device comprises a shaft having a proximal end and a distal end, an engagement member at the distal end constructed and arranged for engagement with the tool; and a locking member at the proximal end constructed and arranged for selectively locking a position of the elongated structure in a manner to maintain a tension on the elongated structure, and unlocking the implanted structure to permit an adjustment of the tension. The engagement member is engaged with the tool, and a proximal end of the elongated structure is inserted through the distal end of the sheath such that a length of the elongated structure extends beyond the proximal end of the sheath. A tension is established on the extended portion of the elongated structure by passing the portion through the locking member, and the locking member is activated to maintain the tension. The tool is activated for initiating a movement of the sheath in the vessel to free the implanted elongated structure from an obstruction encountered by the sheath, and the sheath is advanced into the vessel. The locking member is reactivated, and the tension is adjusted on the elongated structure.

### BRIEF DESCRIPTION OF THE DRAWINGS

FIG. 1 is a perspective view of prior art tool for removing an implanted cardiac lead from a body vessel;

FIG. 2 is a perspective view of one embodiment of a tension control device according to the present invention;

FIG. 3 is a side elevational view illustrating the tension control device of FIG. 2 engaged with a trigger handle of a lead removal tool;

FIG. 4 is a longitudinal sectional view of the tension control device of FIG. 2;

FIG. 5 is an enlarged view of the locking member of the tension control device;

FIG. 6 illustrates a perspective view of the inventive tension control device engaged with a lead removal tool as in FIG. 3, and illustrating the device as it exerts tension on a cardiac lead;

FIG. 7 illustrates a perspective view of another embodiment of a tension control device;

FIG. 8 illustrates a perspective view of yet another alternate embodiment of a tension control device; and

FIG. 9 illustrates a perspective view of the tension control device of FIG. 8 engaged with a lead removal tool.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

The present invention relates to a device for controlling tension on an elongated implanted structure during a procedure for removal of the elongated structure from a body vessel. In the following discussion, the terms “proximal” and “distal” will be used to describe the opposing axial ends of the tension control device, as well as the axial ends of various component features of the device. The term “proximal” is used in its conventional sense to refer to the end of the device (or component thereof) that is closest to the operator during use of the device. The term “distal” is used in its conventional sense to refer to the end of the device (or component) that is at the greatest distance from the operator, or that is initially inserted into the patient.

Among other potential uses, the tension control device may be used in combination with a tool for removing an elongated implanted structure. One example of an implanted structure that can be removed utilizing the inventive device is a cardiac lead. A cardiac lead, as the term is used herein, refers to a lead that is used in connection with a heart-related device. Non-limiting examples of cardiac leads include pacemaker leads, defibrillator leads, coronary sinus leads, and left ventricular pacing leads. In addition to cardiac leads, other indwelling structures targeted for removal may include neurological pacing and stimulation leads, as well as various other implanted catheters, sheaths, cannulae and the like. For convenience, the following discussion will describe the use of the inventive tension control device for removal of a cardiac lead, such as a pacemaker or a defibrillator lead. However it should be understood that this is not intended to be a limitation on the scope of the invention.

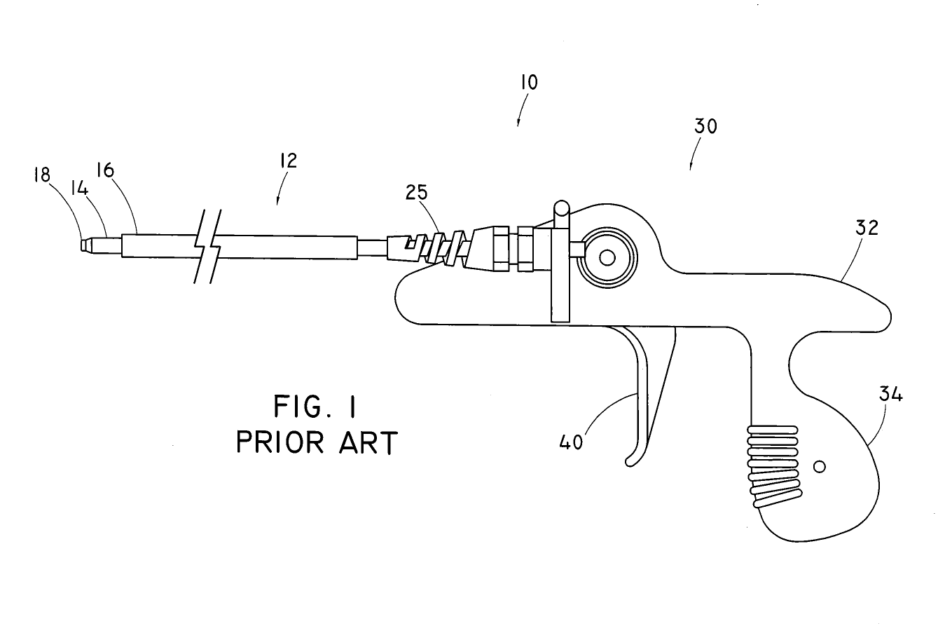

FIG. 1 is a perspective view of one type of a prior art tool 10 for removing an elongated implanted structure from a body vessel. Tool 10 is useful for providing access to the interior of the body vessel to remove the implanted structure, such as a cardiac electrical lead. Prior art removal tool 10 includes a sheath 12 comprising inner and outer coaxial sheath members 14, 16, a distal tip 18, and an optional handle 30. In the prior art tool shown, distal tip 18 is affixed to the distal end of inner sheath member 14. Handle 30 includes a main body portion 32, and an ergonomically shaped grip 34 extending downwardly therefrom. The handle may be equipped with a power source (not shown) to provide rotary and/or axial action to the sheath, or alternatively, may be structured such that the user may manually rotate and/or advance the sheath into the vessel.

Removal tool 10 may be structured such that activation of the power source, or initiation of the mechanical action, may be initiated by grasping the grip 34 and pulling trigger 40 in well-known fashion. Although the prior art tool of FIG. 1 includes inner and outer sheath members 14, 16, the presence of dual sheath members is optional, and the tool may alternatively comprise a single sheath member. In either event, the inner sheath member, or the sole sheath member if only a single sheath is provided, has a lumen dimensioned to receive therein the elongated structure targeted for removal. An optional strain relief 25 may be provided at the proximal end of sheath 12 to inhibit kinking of the sheath during use of tool 10.

During manual operation of prior art tool 10, the distal end of the sheath is inserted over an exposed end of the lead, or the proximal end of a locking stylet. As well known by those skilled in the art, a locking stylet is often used for removal of a lead having a lumen extending therethrough. The distal end of the stylet is inserted as far as possible into the lead, at which point it “grasps” the interior of the lead. As the stylet is withdrawn in the proximal direction, the lead is withdrawn in tandem with the stylet. Further discussion of the structure and operation of a locking stylet is provided in the incorporated-by-reference U.S. Pat. No. 4,988,347.

The operator initially pulls trigger 40 to activate tool 10. This action drives, or translates, the linear motion of the trigger pull to result in rotary motion of sheath 12, and more particularly, inner sheath member 14. As a result, the sheath may be advanced over the lead and into the vessel. As the sheath advances (or, “tracks”) over the lead, distal tip member 18 disengages the lead from obstructions encountered in the vessel, such as encapsulating fibrous material that may have grown around the lead. In the prior art tool 10 illustrated in FIG. 1, the linear motion of the trigger pull may be translated into the rotary motion of inner sheath member 14 via a conventional rack and gear structure (not shown) provided in the handle.

The remaining features of the translation mechanism are not pertinent to the present invention, and need not be further explained or illustrated to enable one skilled in the art to understand the features of the present invention. Those skilled in the art will appreciate that the rack and gear structure described above is exemplary only, and that there are numerous other manual ways in which a device can be structured such that an action generated by an operator, such as a trigger pull, may be translated to rotary or axial (e.g., pulsed) motion for driving the shaft of a device as described. Such conventional techniques are generally suitable for use with the inventive tension control device.

As an alternative to the manual structures described above, the prior art tool 10 may be provided with a power source, such as a drive motor (not shown). The power source may comprise any conventional source suitable for driving the rotation of the sheath, such as a source for generating electrical, battery or pneumatic power. Those skilled in the art can readily select a suitable power source for this purpose.

Further description of prior art removal tool 10, and its operation, is provided, e.g., in the incorporated-by-reference U.S. Pat. Publ. Nos. 2006/0235431, 2008/0071341 and 2008/0071342. Those skilled in the art will appreciate that the following discussion of the tension control device of the present invention will also apply to use of the inventive device with other prior art removal tools, and that removal tool 10 is illustrated and described herein solely to provide one example of a device suitable for use with the inventive tension control device, and not by way of limitation.

As the sheath is advanced over the lead or stylet, it is desirable to maintain a tensional force on the lead. As stated, maintaining this tensional force compels the sheath to follow and track-over the lead within the vessel in a manner that limits undesired progression of the sheath toward, into, and potentially through the vessel wall.

During advancement of the sheath over the lead, one of the hands of the physician is typically occupied by directing the distal end of the sheath(s) over the exposed end of the lead or the stylet, through the vessel opening, and along the path of the lead within the vessel. The other hand is typically occupied by actuating or otherwise operating the removal apparatus (e.g., laser, RF, or mechanical removal device). Therefore, a third hand is usually required in order to apply the tensional force on the lead to allow for safe advancement of the sheath. However, this necessitates the presence of additional trained medical personnel to grasp the end of the lead or stylet, and to apply the tensional force thereon. As stated previously, utilizing another person in the lead removal procedure removes a certain element of control from the primary physician, and increases the cost and complexity of the procedure.

The tension control device of the present invention provides the physician with the ability to control the tension on the lead or locking stylet during the removal procedure, at the same time that the physician carries out the other actions described above. As a result, there is no necessity to provide a second person to apply the tensional force on the lead or stylet.

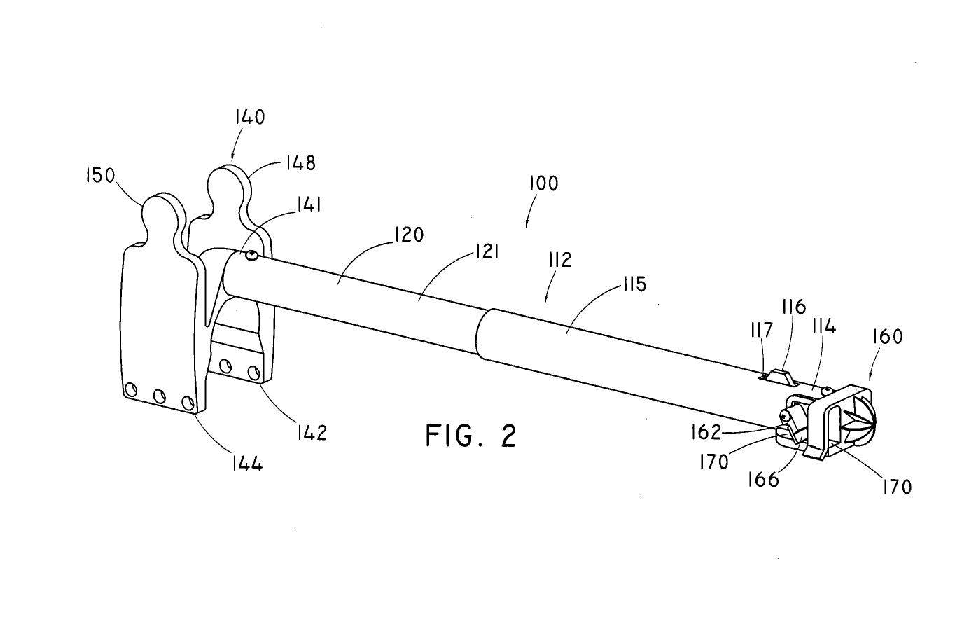

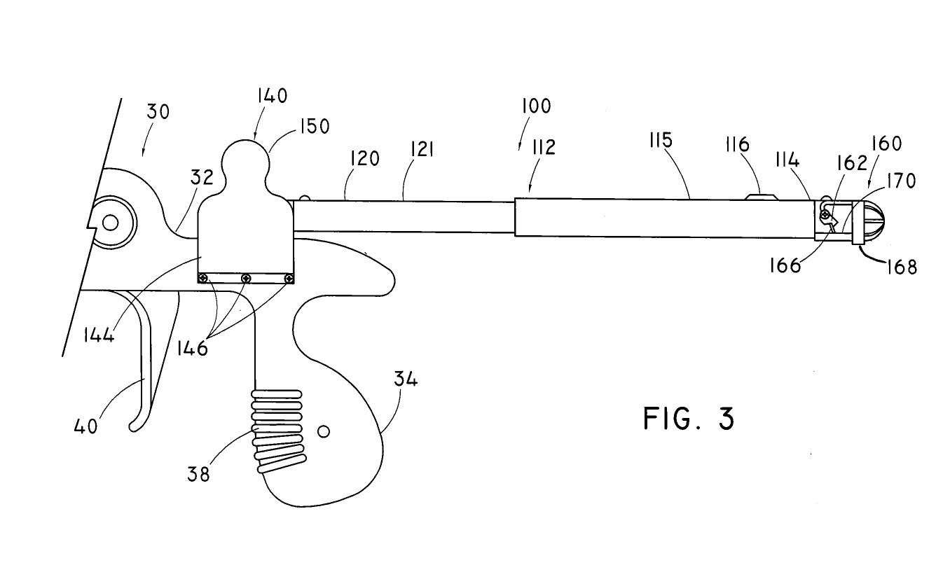

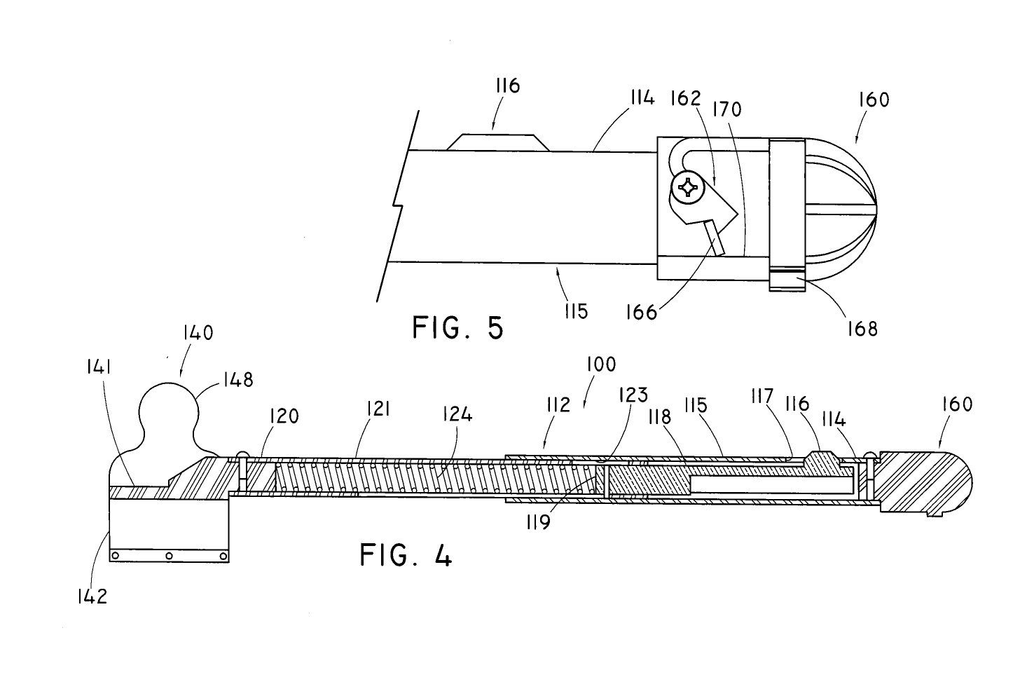

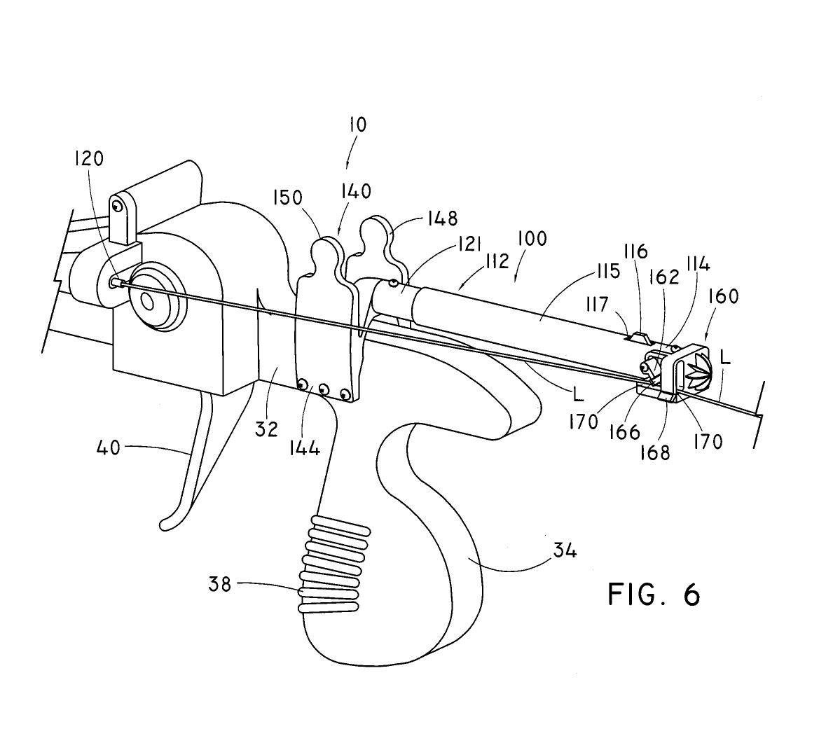

FIG. 2 illustrates a perspective view of one embodiment of a tension control device 100 according to the present invention. FIG. 3 illustrates a side elevational view of the tension control device of FIG. 2 engaged with the handle of prior art lead removal tool 10. FIG. 4 is a longitudinal sectional view of the tension control device 100 of FIG. 2. FIG. 5 is an enlarged view of locking member 160. FIG. 6 is a perspective view of tension control device 100 engaged with the prior art lead removal tool as in FIG. 3, and illustrating the device as it exerts tension on a cardiac lead L.

In the embodiment shown in FIGS. 2-6, tension control device 100 comprises a shaft 112 having a proximal end 114, a distal end 120, an engagement member 140 at distal end 120 for engaging tension control device 100 with a lead removal tool 10, and a locking member 160 at proximal end 114 for releasably locking a lead or a stylet in a manner to control the tension along the length of the lead or stylet.

In this embodiment, shaft 112 comprises first and second telescopically-arranged tubular members 115, 121, each having an inner passageway extending therethrough. A biasing member, such as coiled spring 124 (FIG. 4), is disposed within the inner passageway of (inner) tubular member 121. A slidable boss 118 is disposed within the inner passageway of (outer) tubular member 115. An activating member, such as slide latch 116, extends upwardly through opening 117 in tubular member 115.

Shaft 112 is illustrated in FIGS. 2-4 in a first, or expanded, condition. In this first condition, spring 124 is expanded such that tubular shaft 112 comprises a maximum length. Upon actuation of tension control device 100, slide latch 116 is depressed by the user. Tubular member 115 is then advanced in a distal direction, or “telescoped”, over tubular member 121, until slide latch 116 is received in an opening 123 (FIGS. 4, 6) disposed along the length of tubular member 121. During this movement, leading end 119 of slidable boss 118 acts to compress spring 124, as shaft 112 is maneuvered into the second, or contracted, condition shown in FIG. 6. Further discussion of this movement is provided hereinafter in the description of the operation of tension control device 100.

In the embodiment shown, an engagement member 140 is provided for engagement with the lead removal tool. For purposes of illustration, and not by way of limitation, engagement member 140 as shown is structured for engagement with the prior art lead removal tool 10 illustrated in FIG. 1. FIGS. 3 and 6 illustrate one manner in which such engagement may be effected.

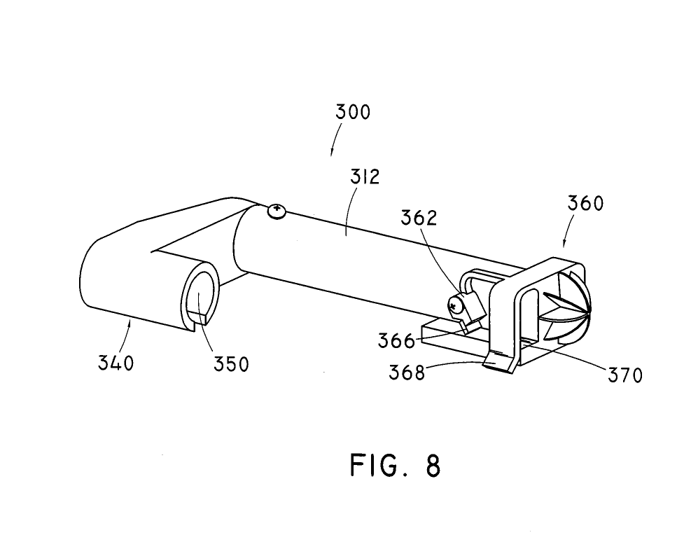

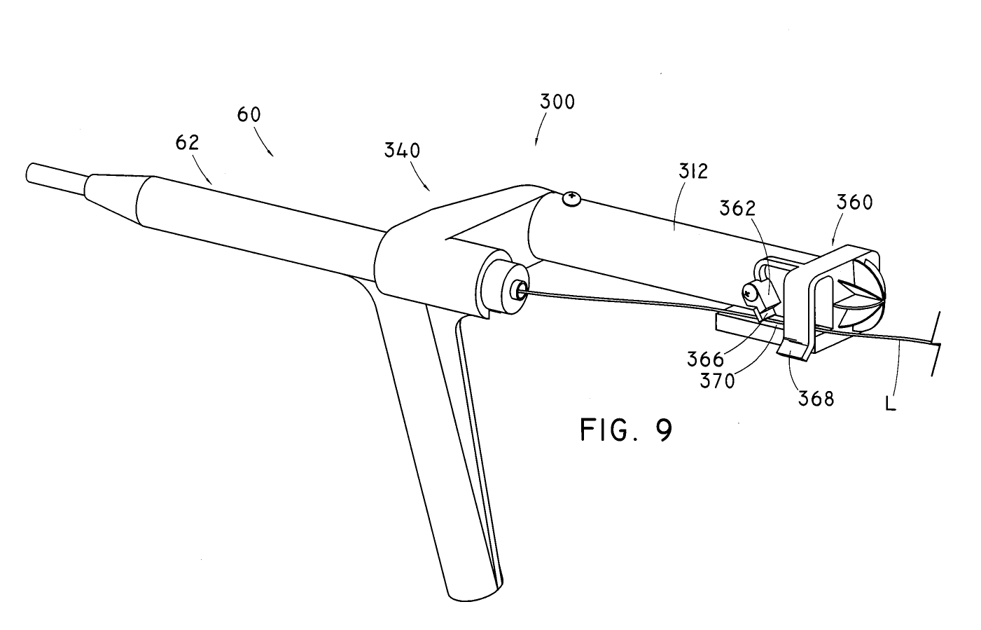

Although the particular configuration of engagement member 140 illustrated herein is preferred in order to facilitate engagement with certain lead removal tools, such as the EVOLUTION® lead removal tool available from Cook Medical, of Bloomington, Ind., this configuration is not required. Rather, engagement member 140 may have any configuration that is capable of providing engagement with a suitable tool, such as lead removal tool 10. One non-limiting alternative is illustrated in FIGS. 8 and 9 by engagement member 340, as further described hereinafter. As still another alternative, tension control device 100 need not necessarily include a discrete engagement member as described, and suitable structure may be substituted for engaging tool 10 and tension control device 100 in a manner in which tension control can be effected as further described herein.

Engagement member 140 includes a body member 141. First and second legs 142, 144 extend in a generally downward direction from body 141. Wings 148, 150 extend upwardly from body 141. Legs 142, 144 are sized for engagement with opposing sides of tool main body portion 32 (FIG. 3). In the embodiment shown, legs 142, 144 include structure for engagement with the opposing sides of tool body portion 32, such as screws 146. Screws 146 may be sized to be received in corresponding apertures in tool main body portion 32, however this arrangement is not necessary in all instances and alternative structure capable of facilitating engagement with a tool may be substituted. Screws 146 as shown herein are merely intended to provide additional gripping force against tool body portion 32 to prevent an inadvertent and unintended disengagement of engagement member 140 from the lead removal tool.

Those skilled in the art will appreciate that any cooperating surfaces, or structure, of the lead removal tool and the engagement member 140 may be substituted for that illustrated herein, the intention merely being to provide sufficient gripping force to maintain the relative position between the engagement member 140 and the tool body portion 32 during use of tension control device 100. Preferably, the engagement will also allow for easy disengagement following removal of the lead from the vessel. In the embodiment shown, engagement member 140 may be disengaged by unscrewing screws 146 (if they have been screwed into body portion 32), or squeezing together wings 148, 150 (if not screwed into the body portion). Squeezing wings 148, 150 in this manner increases a distance between legs 142, 144, thereby facilitating disengagement from body portion 32.

Locking member 160 is positioned at the proximal end 114 of tubular member 115. FIG. 5 illustrates an enlarged view of locking member 160. In the embodiment shown, locking member 160 includes a cam lock holder 162, having a cam lock 166 affixed thereto. Preferably, cam lock holder 162 is biased, such as by spring-loading holder 162, so that cam lock holder 162 and cam lock 166 yield in the proximal direction to permit passage of a length of the elongated structure therethrough (FIG. 6), and return to a starting, or locking, position on a more distal length of the elongated structure to maintain a tension thereon. A locking platform 170 is provided for receiving cam lock 166, and for receiving the elongated structure, e.g., a cardiac lead. Those skilled in the art will appreciate that other locking members having the capability of providing a locking function may be substituted.

An explanation of the operation of tension control device 100 will now be provided. The following explanation will discuss use of the inventive tension control device with the prior art lead removal tool 10 shown in FIG. 1. Those skilled in the art will appreciate that the inventive tension control device may also be used with other lead removal tools.

Initially, engagement member 140 of the tension control device is engaged with lead removal tool 10. For purposes of illustration only and not by way of limitation, FIGS. 3 and 6 show engagement member 140 engaged with body portion 32 of prior art removal tool 10.

In order to arrange prior art lead removal tool 10 for operation, the proximal end of the elongated structure (lead) targeted for removal is inserted through the distal end of sheath 12 of prior art lead removal tool 10 in well-known fashion. The lead is threaded through sheath 12 such that the lead proximal end L extends out the proximal end of the sheath. Slide latch 116 of tension control device 100 is depressed, and tubular member 115 is telescoped over tubular member 121 until the slide latch is received in opening 123 in tubular member 121. As a result of this action spring 124 is compressed, and shaft 112 is maintained in the contracted condition shown in FIG. 6.

In the figures and corresponding discussion, the elongated structure targeted for removal is referred to as lead L. Although the implanted elongated structure targeted for removal as shown in FIG. 6 is referred to herein as a lead for purposes of discussion, the exposed portion of the lead shown in the figure could also comprise another implanted elongated structure, or could comprise a portion of a locking stylet or a lead extender that has been affixed to a proximal end of a lead in well-known fashion. In the case of a locking stylet, the distal end of the stylet will have previously been inserted into the lumen of the lead targeted for removal in conventional fashion. In the case of a lead extender, the distal end of the extender will have been affixed in any conventional fashion with the proximal end of the lead. Thus, even though the elongated structure to which tension is applied will be referred to for convenience in this discussion as lead L, it is intended that a reference to lead L made herein may also refer to an implanted elongated structure, or to a locking stylet or a lead extender applied to a proximal end of a severed lead, and extending in a proximal direction therefrom. In any of the foregoing scenarios, the tension control features of the present invention operate in similar fashion.

As stated previously, the physician's hands are typically occupied during the removal procedure in carrying out the operation of the lead removal tool, and directing the tip of the tool in desired fashion in the vessel. Thus, in a conventional lead removal procedure, the presence of an additional person is usually required, so that the additional person can apply the tension to the proximal end of the lead.

During use of the inventive tension control device, the proximal end of lead L is pulled in a proximal direction to establish a tension on the lead. In order to maintain, or lock, the tension in the lead, lead L is passed through locking member 160. Typically, the lead is inserted into the locking member laterally between outwardly extending finger 168 and locking platform 170, such that the lead rests upon the locking platform. Finger 168 and locking platform 170 are best shown in FIGS. 5 and 6. When lead L is inserted as described, spring-loaded cam lock holder 162, and more particularly, cam lock 166 yield to allow easy insertion of the lead along platform 170. Once the operator ceases pulling on the proximal end of lead L, an internal spring provides spring tension to urge cam lock holder 162, and cam lock 166, to return to the “locking” position shown in FIGS. 5 and 6. As shown in FIG. 6, cam lock 166 lockingly engages lead L, thereby maintaining a tension on lead L, and inhibiting undesired axial movement of the lead.

Latch 116 is now depressed to release spring 124 from the compressed position, and thereby, to disengage shaft 112 from the contracted condition shown in FIG. 6. Once released, spring 124 exerts a biasing force against tubular member 115, thereby urging tubular member 115 in a proximal direction. However, since the distal end of lead L is bound to encapsulating tissue within the body vessel, the force exerted by spring 124 is typically not of sufficient strength to overcome the binding force exerted on the lead by the encapsulating tissue. As a result, only minimal, if any, expansion of the length of shaft 112 can occur.

The lead removal tool is activated to sever the encapsulating tissue, such as by squeezing trigger 40 of the prior art lead removal tool 10 illustrated herein. This action causes rotation or a similar advancing motion of inner sheath member 14, and particularly, of sheath distal tip 18. As distal tip 18 rotates, it cuts, cores, disrupts, or otherwise advances through encapsulating tissue or other obstructions that are encountered in the vessel. Repetitive squeezing of the trigger enables the operator to slowly advance distal tip 18 further into the vessel, thereby freeing the lead from the obstructions as the sheath continues to advance.

As the sheath 12 is slowly advanced relative to the lead, the length of the lead traversed by the sheath is slowly freed from the encapsulations and obstructions. As a length of the lead within the vessel becomes freed, the force exerted by spring 124 on tubular member 115 is now able to overcome the resistance previously provided by the encapsulated length of the lead. As a result, tubular member 115 is urged in the proximal direction, thereby expanding the length of shaft 112. Generally, the length of shaft 112 increases a length roughly equal to a distance that the sheath has advanced along the lead. As sheath 12 is further advanced into the vessel and additional lengths of the lead are freed from encapsulation, the spring causes further expansion of shaft 112. As the length of shaft 112 expands, the sheath is able to maintain tension on the lead.

Once the shaft 112 reaches its maximum length as shown, e.g., in FIG. 3, it can extend no further to maintain the tension on lead L during additional advancement of the sheath. Accordingly, as advancement of the sheath is continued, the tension exerted on the exposed length of the lead will be relaxed. In order to maintain, or re-establish a tension on the lead, the physician may simply re-load the tension control device 100. In this event, tubular member 115 is once again telescoped over tubular member 121 until slide latch 116 is received in opening 123 of tubular member 121. Tension can be re-established on lead L, the slide latch can be depressed, and further advancement of the lead removal tool in the vessel can then be resumed as described previously. This action can be repeated as many times as necessary until the lead is sufficiently freed from obstructions to permit removal of the lead from the vessel.

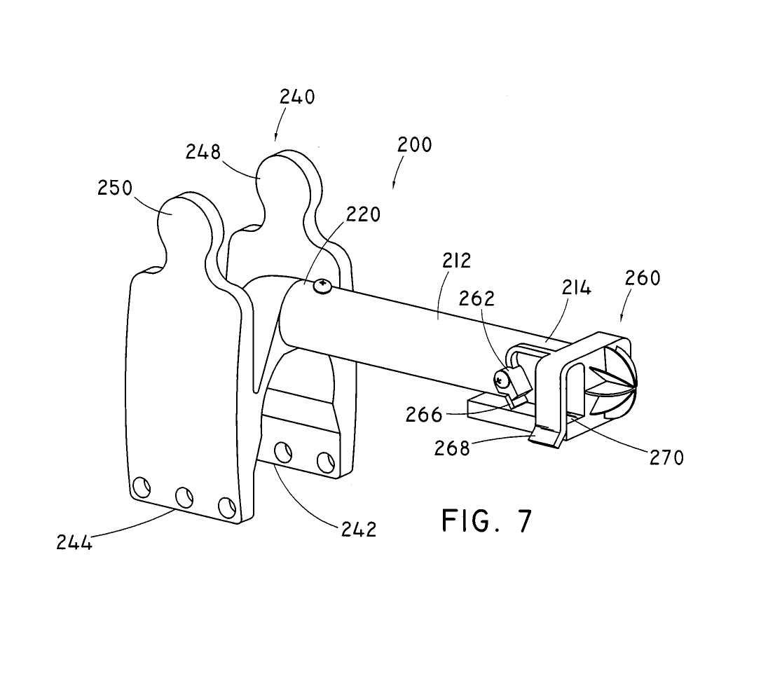

An alternative embodiment of a tension control device 200 is illustrated in FIG. 7. Tension control device 200 is similar in many respects to tension control device 100. In the embodiment shown, tension control device 200 comprises a shaft 212 having a proximal end 214 and a distal end 220. An engagement member 240 is provided at distal end 220 for engaging tension control device 200 with a lead removal tool, and a locking member 260 is provided at proximal end 214 for releasably locking a lead or a stylet.

In this embodiment, engagement member 240 includes first and second downwardly-extending legs 242, 244, structured for engagement with the removal tool. Upwardly-extending wings 248, 250 may be provided to facilitate disengagement from the removal tool. Locking member 260 includes a spring-loaded cam lock holder 262, having a cam lock 266 affixed thereto, as well as a lead-receiving finger 268 and a locking platform 270, all in the same manner as tension control device 100.

Unlike tension control device 100, the shaft 212 of tension control device 200 has a fixed length. Shaft 212 does not include telescopically arranged tubular members, nor does it include a coiled spring or other structure for exerting a tension to increase the length of the shaft.

In operation, tension control device 200 may be engaged with the lead removal tool via engagement member 240 in the same manner as described above with regard to tension control device 100. Similarly, the lead targeted for removal is threaded through the sheath 12, and a tension in the lead is established on the lead by pulling it in the proximal direction. Tension may be maintained on the lead by utilizing a locking mechanism, such as locking member 260, in the same manner as described above.

The lead removal tool 10 is activated by squeezing trigger 40, thereby causing the rotation and advancement of sheath distal tip 18. Once again, repetitive squeezing of the trigger slowly advances distal tip 18 further into the vessel, and frees the lead from the obstructions encountered therein.

As the physician advances sheath 12 further into the vessel, the tension on the lead is relaxed, and the exposed portion of lead L begins to sag. Since it is often desirable to maintain a tension on the lead as illustrated in FIG. 6, the physician may now manipulate the lead control device 200 to re-establish this tension. In this event, the physician may simply pull on the proximal end of lead L (the portion of lead L extending to the right of tension control device 100 in the orientation of FIG. 6), thus re-establishing a tension on the lead. As lead L is being pulled by the physician, the spring-loaded cam lock holder 262 and cam lock 266 yield to permit the lead to extend in the proximal direction along locking platform 270. Once the desired tension is re-established, the physician releases the pulling force on the lead, and spring-loaded cam lock holder 262 and cam lock 266 are biased back to the locking position on the lead, e.g., as shown in FIG. 6. This action can be repeated as many times as desired during the lead removal procedure to free the lead from obstructions in the body vessel.

FIG. 8 illustrates another embodiment of a tension control device 300. Tension control device 300 is similar in many respects to tension control devices 100, 200. Tension control device 300 has a fixed length shaft 312, in the same manner as fixed length shaft 212 of device 200. Tension control device 300 is provided with an engagement member 340 that is shaped to facilitate engagement with a lead removal tool of a particular configuration, and a locking member 360 for exerting tension on a lead L.

Engagement member 340 is shaped for affixation to a lead removal tool. Although engagement member 340 can be shaped to receive a lead removal tool such as tool 10, in this embodiment engagement member 340 is shaped to have an annular passageway 350 extending therethrough. Passageway 350 is particularly shaped and sized to receive a shaft, or a sheath, of a correspondingly shaped and sized lead removal tool. FIG. 9 illustrates engagement member 340 engaged with a prior art lead removal tool 60. In this example, lead removal tool 60 is of a type having a generally annular shaft 62. Some lead removal tools, particularly tools having a power mechanism, are provided with a shaft that is shaped as shown. The engagement member 340 is shaped in a corresponding manner to the particular shape of shaft 62 of the lead removal tool 60 to enhance engagement therebetween.

Although the engagement member 340 is shaped for engagement with the prior art tool 60 as shown, this is merely one example of a particular configuration that can be provided to the engagement member. Those skilled in the art will appreciate that the configuration of the engagement member 340 may be modified in any way that will enhance engagement with the particular configuration of a tool.

In this embodiment, shaft 312 is offset from the engagement member. When a tension control device is configured as shown for engagement with an annular sheath 62 of a lead removal tool as shown in FIG. 9, providing a shaft 312 offset from the engagement member 340 and locking member 360 improves the line of sight of the physician. Similarly, providing an offset shaft as illustrated may improve the ability of the physician to grip the shaft.

In the embodiment shown, locking member 360 is structured in the same manner as the locking members of tension control devices 100 and 200. Accordingly, locking member 360 includes a spring-loaded cam lock holder 362 having a cam lock 366 affixed thereto, a lead-receiving finger 368, and a locking platform 370. Operation of tension control device may be carried out in the same manner as described above with regard to tension control device 200.

Those skilled in that art will appreciate that the foregoing detailed description should be regarded as illustrative rather than limiting, and that it should be understood that it is the following claims, including all equivalents, that are intended to define the spirit and scope of this invention.

1. A device for controlling a tensional force on an implanted elongated structure during removal of the implanted elongated structure from a body vessel of a patient via a removal tool, said removal tool comprising a sheath member having a passageway extending therethrough, said sheath member sized such that the elongated structure is receivable in said passageway and a proximal end of the elongated structure is extendable therethrough, the device comprising:
a shaft member structured for engagement with said removal tool, said shaft member having a locking member disposed thereon, said locking member constructed and arranged for selectively locking a position of the elongated structure in a manner to maintain a tension on the elongated structure, and unlocking the elongated structure to permit an adjustment of said tension.

2. The device of claim 1, wherein said shaft member comprises a proximal end and a distal end; an engagement member positioned at said shaft distal end for effecting said engagement with said removal tool;
said locking member positioned at said shaft proximal end.

3. The device of claim 2, wherein said shaft member comprises an elongated body comprising first and second tubular members, each of said first and second tubular members having a proximal end and a distal end, and having a passageway extending longitudinally therethrough; said tubular members telescopically arranged such that one of said tubular members is receivable within the passageway of the other tubular member.

4. The device of claim 3, wherein said proximal end of said first tubular member has said locking member disposed thereat, and said distal end of said second tubular member has said engagement member disposed thereat, said second tubular member being sized to be received within the passageway of the first tubular member, said second tubular member having a biasing member positioned in said second tubular member passageway, and said first tubular member having a boss member positioned in said first tubular member passageway, said boss member sized and positioned for contracting a length of said biasing member as said second tubular member is received within the passageway of the first tubular member.

5. The device of claim 4, further comprising an activating member in engagement with said boss member, said activating member positionable with reference to said boss member for maintaining a position of said boss member within said first tubular member passageway, and actuable for allowing axial movement of said boss member relative to said biasing member for contracting said biasing member length, said activating member further being positionable for maintaining said contracted biasing member length.

6. The device of claim 5, wherein each of said first and second tubular members comprises an opening along a length of said respective tubular member, and said activating member comprises a slide latch extendable through said first tubular member opening, said slide latch initially actuable for allowing said boss member axial movement, and extendable through said second tubular member opening for maintaining said contracted length.

7. The device of claim 2, wherein said engagement member is constructed for releasable engagement with said removal tool.

8. The device of claim 1, wherein said locking member comprises a cam lock for maintaining said tension on the elongated structure.

9. The device of claim 8, wherein said cam lock is spring-actuable for maintaining said tension.

10. The device of claim 2, wherein at least one of said engagement member and said locking member is axially offset from said shaft member.

11. The device of claim 10, wherein both said engagement member and locking member are axially offset from said shaft member.

12. A system for removing an elongated cardiac lead from a body vessel, the system comprising:
a lead removal tool comprising an elongated sheath having a proximal end, a distal end, and a passageway extending therethrough, the sheath having a length such that at least a distal portion of the sheath is receivable in the body vessel, the passageway sized such that the cardiac lead is receivable therein and a proximal end of the cardiac lead is extendable therethrough; and
a tension control device engaged with the lead removal tool, said tension control device comprising a shaft member having a proximal end and a distal end; an engagement member at said distal end, said engagement member constructed and arranged for engagement with the lead removal tool; and a locking member at said proximal end, said locking member constructed and arranged for receiving said extended cardiac lead proximal end and selectively locking a position of the cardiac lead in a manner to maintain a tension on the lead, and unlocking the position of the cardiac lead to permit an adjustment of said tension.

13. The system of claim 12, wherein said locking member comprises a cam lock having a bias member for maintaining said tension on the cardiac lead.

14. The system of claim 12, wherein at least one of said engagement member and said locking member is axially offset from said shaft member.

15. The system of claim 14, wherein both said engagement member and locking member are axially offset from said shaft member.

16. A method for controlling tension on an implanted elongated structure during removal of the implanted elongated structure from a body vessel of a patient, comprising:
providing a tool for removing the implanted elongated structure, said tool comprising a sheath having a proximal end, a distal end, and a passageway extending therethrough, said sheath sized such that the elongated structure is receivable in said passageway;
providing a device for engagement with said tool, said device comprising a shaft having a proximal end and a distal end, an engagement member at said distal end, said engagement member constructed and arranged for engagement with said tool; and a locking member at said proximal end, said locking member constructed and arranged for selectively locking a position of the elongated structure in a manner to maintain a tension on the elongated structure, and unlocking said implanted structure to permit an adjustment of said tension;
engaging said engagement member with said tool;
inserting a proximal end of said elongated structure through the distal end of the sheath such that a length of the elongated structure extends beyond the proximal end of the sheath;
establishing a tension on the extended portion of the elongated structure by passing said portion through said locking member in a manner to achieve said tension, and activating said locking member to maintain said tension;
activating said tool for initiating a movement of said sheath in said vessel to free said implanted elongated structure from an obstruction encountered by said sheath, and advancing said sheath into said vessel; and
re-activating said locking member and adjusting said tension on said elongated structure.

17. The method of claim 16, wherein said tool activating step and said locking member re-activating step are repeated as said sheath is advanced into said vessel.

18. The method of claim 16, wherein said locking member comprises a cam lock for maintaining said tension on the elongated structure.

19. The method of claim 18, wherein said cam lock is spring-actuable for maintaining said tension.

20. The method of claim 16, wherein at least one of said engagement member and said locking member is axially offset from said shaft member.
